(19) United States
(12) Patent Application Publication
(10) Pub. No.: US 2005/0049608 A1
(43) Pub. Date: Mar. 3, 2005

(54) Stent delivery catheter

(76) Inventors: Harold M. Aznoian, North Andover, MA (US); Michael F. Weiser, Groton, MA (US)

Correspondence Address: KIRKPATRICK & LOCKHART LLP, 75 STATE STREET, BOSTON, MA, 02109-1808, US

(21) Appl. No.: 10/867,428

(22) Filed: Jun. 14, 2004

Related U.S. Application Data
(60) Provisional application No. 60/478,050, filed on Jun. 12, 2003.

(51) Int. Cl. A61F 11/00

(52) U.S. Cl. 606/108

(57) ABSTRACT

A delivery device for a biliary stent constructed of thermoplastic material with ridges and valleys formed along the outer surface of the stent that provide a helical, thread-like configuration. The delivery device comprises an external surface that is selectively expandable to engage an interior surface of the tubular stent. Additionally the exterior surface may include ridges and valleys that coincide with ridges and valleys defined by the helical thread extending through the stent in order to provide a more secure engagement with the stent during delivery.

### RELATED DISCLOSURE INFORMATION

This application claims the benefit of U.S. Provisional Application No. 60/478,050, filed on Jun. 12, 2003, the subject matter of which is related to the disclosure document filed at the U.S. Patent and Trademark Office on Jun. 12, 2001, and assigned Disclosure Document No. 495220. The entire teachings of the application and disclosure are incorporated herein by reference.

### FIELD OF THE INVENTION

This invention relates to stents and stent delivery devices and, in particular, to devices adapted for use in the biliary tract.

Biliary stents, for many years, have been made in the form of a polymer tube that can be advanced on a delivery catheter through an endoscope and into the bile duct where it is deployed. The tubular stent is selected to be sufficiently strong to resist collapse to maintain an open lumen through which digestive liquids can flow into the digestive tract. Among the desirable features of such a stent is that it be longitudinally flexible to be advanced along a path that may include sharp bends. The stent also should maintain its intended position within the bile duct without migrating from that position.

### BACKGROUND OF THE INVENTION

Polymeric tubular stents typically have been placed with a catheter-like device that includes telescoping inner and outer tubes, with the stent being mounted on the distal end of the inner tube and the distal end of the outer tube being in engagement with the proximal end of the stent. After the stent has been advanced and manipulated into the intended deployment site in the duct, the outer tube is maintained in its position while the inner tube is retracted, thereby leaving the stent within the biliary tract. Generally, such stents are provided with a retention member at each of the ends of the stent. Among the more common retention devices is the provision of one or more (four to eight are common) retention tabs formed by making an oblique slit along the length of the tube. Each slit defines a tab and enables the tab to project slightly radially outwardly of the outer surface of the tube to engage the luminal surface of the biliary duct to prevent migration. The tabs at the opposite ends of the stent extend toward the middle of the stent as well as radially outward. The openings defined by the tab-forming skives may provide access to the interior of the stent of cellular or other material that may tend to develop into an obstruction tending to restrict flow through the stent. Also among the difficulties with prior polymeric stents is that in some cases the physician may not be able to push the stent through a constriction in the duct. It is among the general objects of the invention to provide a polymeric stent that displays a combination of significant longitudinal flexibility to facilitate its placement and, significant hoop strength to resist collapse of the stent. It is also among the objects of the invention to provide a new approach to securing the position of the stent within the duct as well as providing improved means by which the stent can be advanced through a tight restriction.

### SUMMARY OF THE INVENTION

The stent is formed from a tube of relatively stiff thermoplastic polymer to include ridges and valleys along its outer surface. The ridges and valleys may be helical and may form a thread-like configuration. The ridges and valleys are formed by thermoplastic deformation of the outer surface of the tube. The dimensions of the ridges and valleys can be varied to provide stents with different characteristics. The proximal and distal ends of the stent are preferably not provided with valleys or ridges. The distal end may be tapered to facilitate its entry into the biliary tract. Additionally, the distal end of the stent, which will serve as an inlet for biliary liquids, may have an elongate shape to provide a wider mouth for entry of such liquids. The device may be placed by pushing it to the desired location in the biliary tree, as is presently done, or in accordance with the invention, the stent can be rotated so that the helical ridges and valleys can serve as threads to advance the stent through a biliary stricture. The ridges engage the walls of the duct to secure the stent in place.

It is among the general objects of the invention to provide an improved stent, particularly for use in the biliary tract. Also among the objects of the invention are to provide a stent for use in the biliary tract in which the stent is easily fabricated from a polymeric material and embodies a construction that enables the characteristics of the stent to be varied easily; to provide a stent that is very flexible yet in which the flexibility can be controlled during manufacture without changing the general structure of the stent and; to provide a stent that can be advanced into place by pushing it into place or by threading it through a biliary stricture.

It is another object of the invention to provide a delivery device for the stent that takes advantage of its helical ridge configuration to provide secure engagement during delivery or withdrawal of the stent.

### DESCRIPTION OF THE ACCOMPANYING DRAWINGS

The foregoing and other objects and advantages of the invention will be appreciated more fully from the following description thereof, with reference to the accompanying drawings wherein:

FIG. 1 is a side view of a stent in accordance with the invention in which portions of the stent are broken away;

FIG. 2 is an enlarged illustration of the presently preferred embodiment of the proximal end of the stent;

FIG. 3 is an embodiment of the distal end of the stent;

FIGS. 4 and 5 are top and side views of the thermo forming tool in engagement with the polymer tubing during formation of the stent;

FIG. 6 is an illustration, as seen along the axis of the starting tube during formation illustrating the manner in which the forming tool may press the starting tube against the outer surface of an undersized mandrel passing through the starting tube; and

FIG. 7 is an illustration of the distal end of an embodiment of the invention seen along the line 7-7 of FIG. 1.

FIG. 8 is a side sectional view of a stent delivery device according to one embodiment of the invention;

FIG. 9 is a side sectional view of a stent delivery device including a wedge and a flexible sock held between the wedge and the stent;

FIGS. 10A and 10B are an isometric views of a stent delivery device comprising an expandable helical spring member to be inserted into the stent as shown in an expanded configuration and a collapsed configuration;

FIGS. 11A and 11B show side views of another stent delivery device comprising an expandable flexible sleeve member shown in an expanded state and a contracted state; and

FIGS. 12A and 12B show isometric views of another stent delivery device comprising an expanded spring coil shown in an expanded state and a contracted state;

FIGS. 13A and 13B show a top view and a sectional side view, respectively, of a delivery device employing a selectively engageable key.

### DESCRIPTION OF THE ILLUSTRATIVE EMBODIMENTS

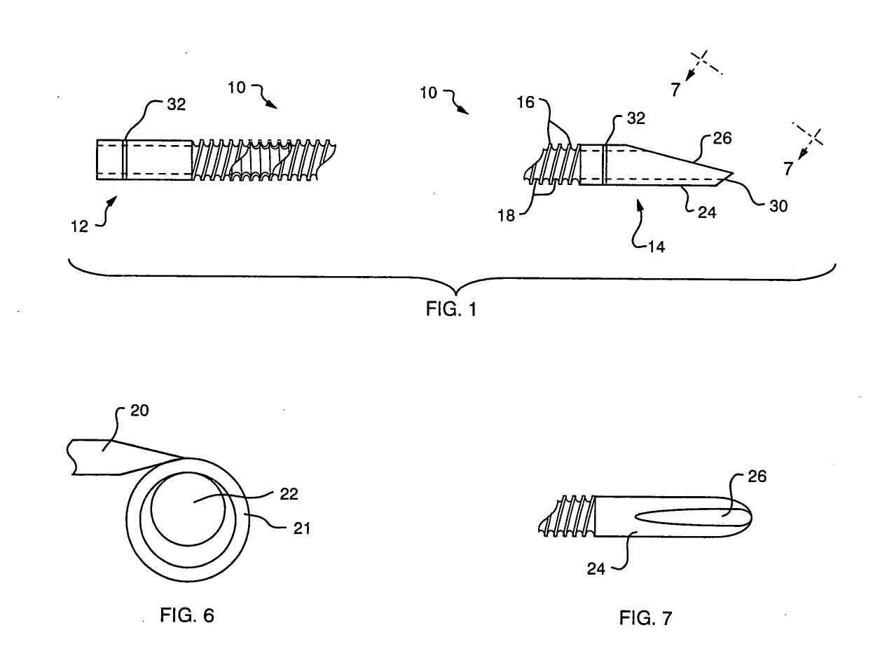

FIG. 1 illustrates, in side view, an embodiment of a stent 10 having a proximal end 12 and a distal end 14. The stent is formed from a tube of a polymer, commercially available from Victrex under the trade designation PEEK. The polymer is a polyetheretherketone, a linear aromatic semi-crystalline polymer. By way of example, for use as a biliary stent, the PEEK tubing may be of the order of 4 to 15 centimeters long having an outer diameter of between about 5 to 11 French (0.065 inches to 0.143 inches). The wall thickness of the tubing may be of the order of about 0.005 inches. The PEEK material is thermoplastic and is formed from its extruded tubular configuration to that illustrated in FIG. 1 in which at least a portion of the length of the tube defines circumferentially extending external ridges 16 alternating with valleys 18. Preferably the ridges are formed in a helical, thread-like pattern.

The ridges 16 and valleys 18 are formed by applying a heated tool against the outer surface of the starting tube while rotating the tube and advancing the tool along the length of the rotating tube. FIGS. 4 and 5 illustrate, diagrammatically, a simplified technique for making the stent in which a generally conically shaped heated tip 20, as might be mounted on the end of a soldering iron, is applied to the external surface of the tubing while the tubing is rotated. The heat and pressure of the thermo forming tool 20 causes the thermoplastic tubing to become flowable in the localized region of the tool, thereby forming the valleys and ridges in the outer surface of the tube. We have found that it is possible to form the stent so that the inner surface of the tube also includes valleys and ridges corresponding to those on the outer surface by initially mounting the starting tube on a mandrel that has an outer diameter smaller than the inner diameter of the PEEK tubing. By way of example, we have found that mounting tubing 21 on a cylindrical mandrel 22 having an outer diameter about 0.010 inches smaller than the inner diameter of the starting tube (see FIG. 6), the configuration of inner and outer ridges and valleys results. It is believed that the inner surface of the tube also forms the ridges and valleys as a consequence of the localized cooling of the polymer immediately behind the axially advancing thermoforming tool. The clearance between the outer diameter of the mandrel and the inner diameter of the tubing is believed to contribute to the ability of the tubing to cool and form in that fashion.

The configuration of the thermo forming tool and the penetration depth to which the tool is applied to the outer surface of the PEEK tube can be varied to vary the characteristics of the stent. Additionally, the speed the tube is rotated and/or the speed the tool is advanced along the length of the tube can also be varied to alter the characteristics of the stent. Deeper grooves 18 may result in a thinner wall having greater flexibility. Similarly, the pitch of the ridges 16 can be varied to vary the characteristics of the stent. As will be understood, increasing the number of threads per unit length of tube will increase the ability to finely adjust the placement of the stent via rotation while decreasing the number of threads per unit length of tube will decrease the ability to finely adjust the placement of the stent. The thread density can thus be adjusted to the particular characteristics of the luminal wall engaged by the stent. By way of example, a relatively rigid luminal wall will allow the use of a densely threaded stent. A relatively flexible or pliable luminal wall will require a stent with less dense and larger threading to ensure the preferably helical threads positively engage the wall to allow for advancement of the stent via rotation.

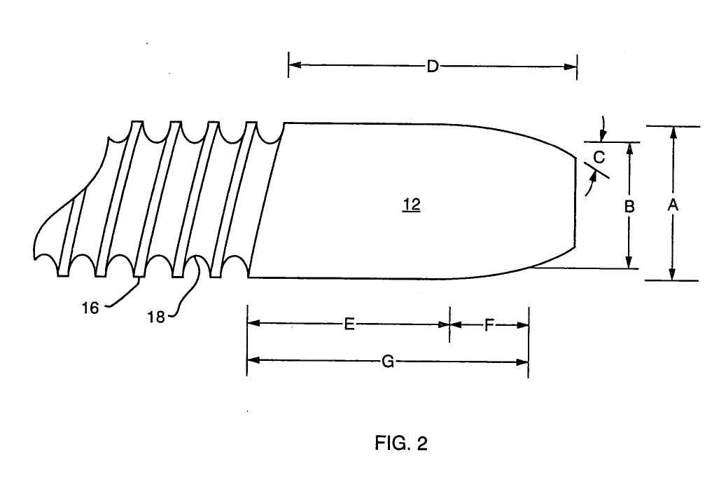

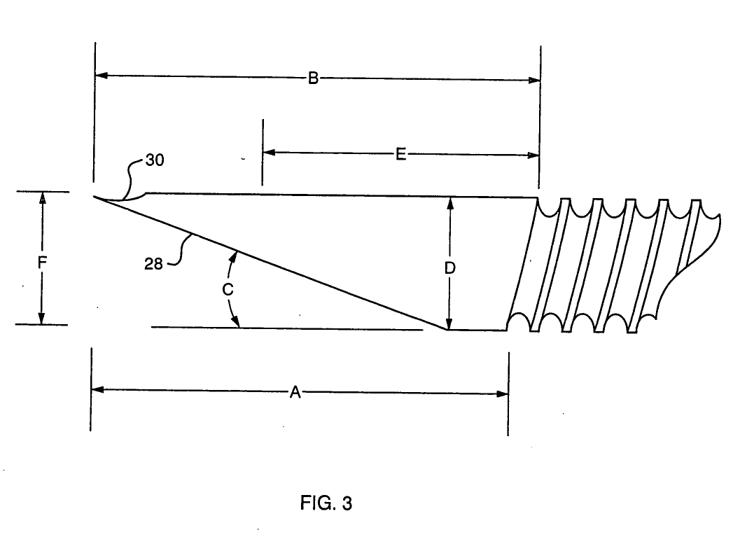

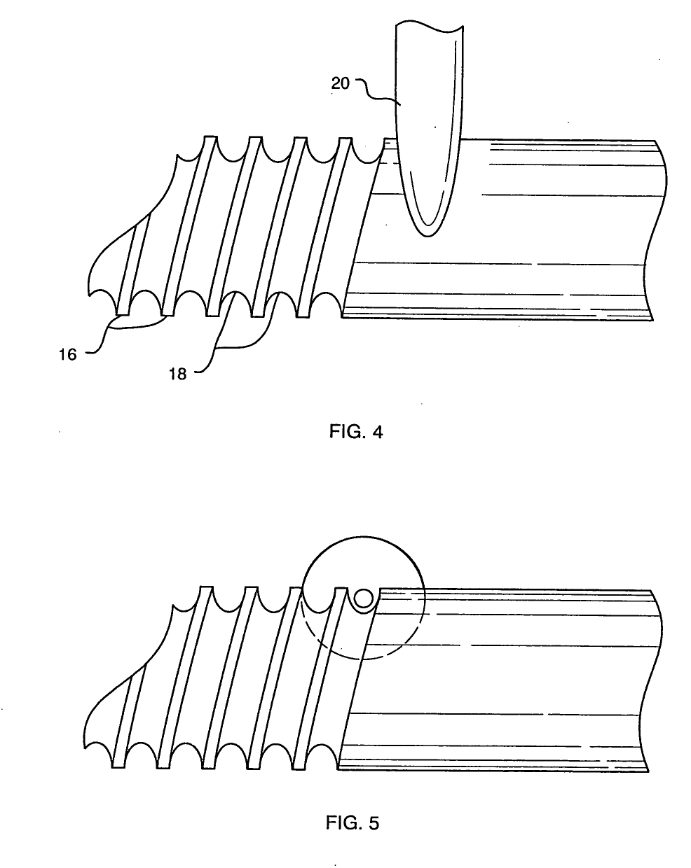

Preferably the proximal end of the starting tube will not have been formed to include the ridges and valleys The proximal end of the stent may be configured and dimensioned as indicated in FIG. 2, in which the end is somewhat radiused or rounded. The rounding may be effected in any number of ways, such as by placing a mandrel having rounded ends within the tube and heating the tube proximal end while rotating the tube to form the rounded end. Another possible approach is to round over the proximal end with a solder tip held against the end while rotating the tube. A yet further approach is to use a knife held at an angle against the proximal end while rotating the tube. The distal end may be provided with a similarly fashioned tip, configured and dimensioned as indicated in FIG. 3. Alternately, it may be preferable to provide a modified tip 24 at the distal end 14 that has a generally tapered configuration to facilitate its entry through the papilla and into the biliary duct. The distal tip also may be configured to provide a distal opening 26 (FIG. 7) that is elongated and may be generally elliptical in shape. The elongated distal opening may facilitate entry of biliary liquids into the stent by providing an inlet opening larger than the transverse cross section of the lumen. To that end, the distal end of the tube is formed with an oblique cut 28 to expose an elongate inlet opening 26. The distal tip also may be beveled or otherwise tapered at its opposite side as shown at 30.

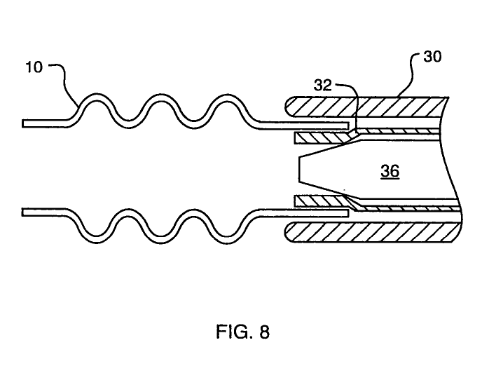

The stent may be provided with marker bands 32 at one or both of the distal and proximal ends. The marker bands 32 may be formed from gold or other suitably radiopaque material. Circular grooves may be thermoformed in either or both of the ends 12, 14 and the radiopaque marker bands may be secured within those grooves. The ridges 16 and grooves 18 may be formed along substantially the full length of the stent or may be formed only along selected segments, for example, adjacent the ends of the stent, leaving the mid portion in its original tubular configuration. Conversely, only the mid portion may be provided with the ridges and valleys. Still further, ridges 16 and grooves 18 can be placed in selected sporadic groups along the length of the stent to achieve stiffness and flexibility characteristics tailored to particular needs or particular anatomy. The valleys and ridges can be made to be circumferentially segmented portions as opposed to being complete annular rings or completely helical by intermittently withdrawing and applying the thermoforming tool. Additionally, the pitch of the ridges and the depth of the valleys can be varied along any segment or along the full length of the stent in order to provide varied flexing characteristics for the stent. The stent may be placed in the biliary duct either by the conventional pushing technique or by mounting it on a rotatable delivery catheter having a stent engaging member engageable with the proximal end of the stent. FIG. 8 shows a catheter 30 with a stent engaging member 32 in the form of an expandable collar that is received within the proximal end of the stent 10 and expanded securely against the inner luminal surface at the proximal end with wedge 36. Stent 10 is advanced to a selected site in the biliary tract with catheter 30. Wedge 30 is then proximally retracted to release the frictional engagement of engaging member 32 from stent 10. Stent 10 is then released using the conventional pushing technique. In an alternate embodiment, as the stent is advanced into the biliary duct, the an alternate delivery device (not shown) may be rotated to facilitate entry of the stent through an obstructed portion of the duct. To that end, it is preferred that the ridges and valleys be formed to define a helical path that will enable the stent to advance, in screw-like fashion through the obstruction. The stent may be released from the delivery device after it has been deployed in the desired location.

The ridges may engage the inner surface of the duct to secure the stent in place. Additionally, when the valley 18 is continuous, as defined by a helical path, it may be possible for biliary liquids to flow between the outer surface of the stent and the wall of the biliary duct as well as through the stent itself. To perform the latter function, valleys 18 have to be formed with sufficient pitch, depth and/or angle to maintain an open channel since it is anticipated the duct wall will partially herniate into the valley. Another benefit of having a relatively deep valley is that such a configuration is expected to enhance the mechanical engagement of the stent to the duct wall.

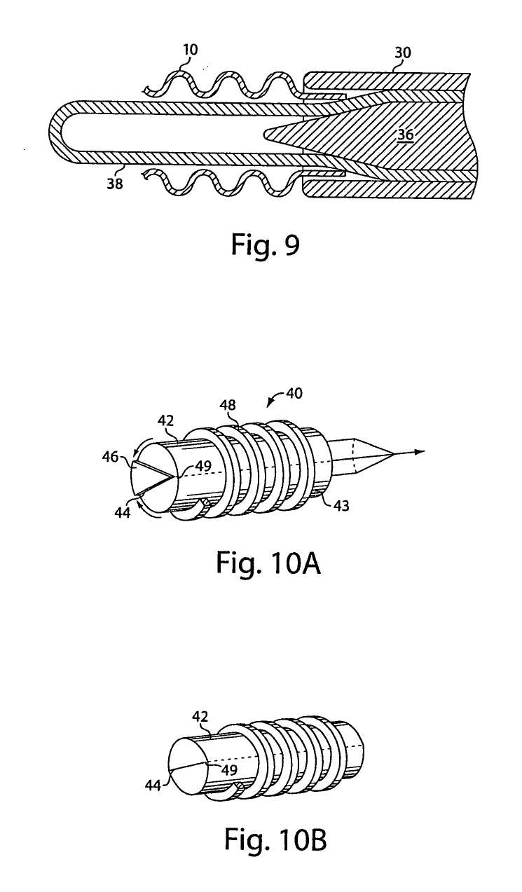

FIG. 9 shows an alternate arrangement for the stent delivery device as shown in FIG. 8. In FIG. 9, the engaging member 32 is configured as a sock formed from a material such as a polymer to help provide frictional engagement between the wedge 36 and the stent 10.

FIGS. 10A and 10B show an alternate stent delivery device 40 comprising an expandable male member configured as a cylinder 42 that is sized to be inserted into the stent 10 to engage its inside surface. The expandable cylinder 42 is configured to have a continuous slot 44 along its length that may be selectively filled with a corresponding pie-shaped wedge 46 to prop open the cylinder in an expanded configuration as shown in FIG. 10A. When the wedge 46 is removed from the cylinder, such as by proximal withdrawal by the operator, the cylinder resiliently collapses to a low profile configuration as shown in FIG. 10B. The cylinder closes resiliently about a living hinge 49 extending along the length of the cylinder 42 opposite the slot 44. To more reliably engage the inner surface of the stent, the cylinder may be provided with a helical ridge 48 along its outer surface 43 that corresponds to the peaks and valleys defined by the helical ridge of the stent (on the inside surface).

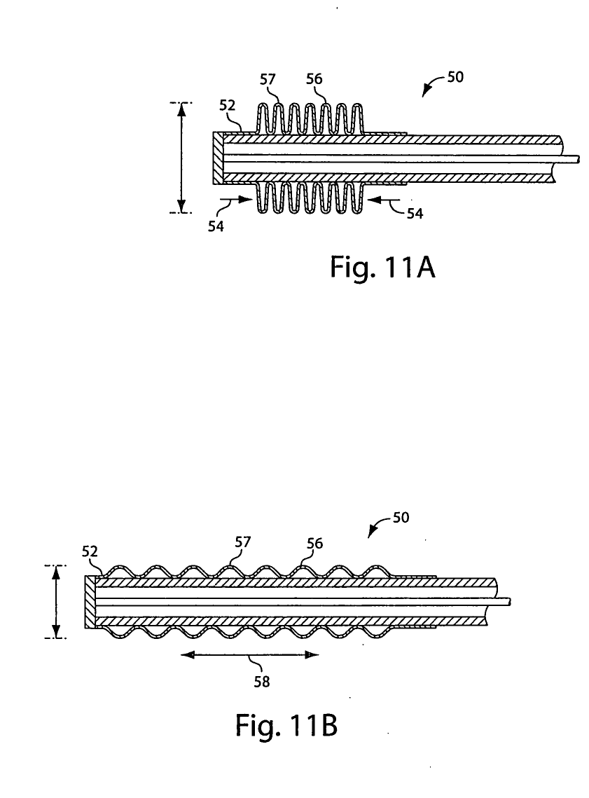

FIGS. 11A and 11B show another stent delivery device 50 in which the catheter comprises a flexible sleeve 52 that is selectively expandable and retractable to engage an inner surface of the stent 10. The flexible sleeve 52 expands to an increased profile that engages the inside surface of the stent when a compressive force (indicated at 54) is applied to its ends. The sleeve collapses, creating a plurality of folds 56 or crinkles that expand radially outward as the longitudinal compressive force is applied and length of the sleeve is compressed as shown in FIG. 11A. The individual peaks of the folds may serve to engage the peaks and valleys of the inside surface of the stent to provide additionally security in engagement. When a longitudinal tensile force 58 is applied to the sleeve 52 as shown in FIG. 11B, the sleeve length increases and the magnitude of the folds 56 decreases correspondingly. In this condition, the sleeve disengages from the inside surface of the stent permitting its release. The configuration and operation of the present embodiment is similar to the delivery device disclosed in U.S. Pat. No. 6,248,112 (Gambale et. al.) commonly assigned to the assignee of the present invention. The disclosure of the '112 patent is incorporated by reference herein in its entirety.

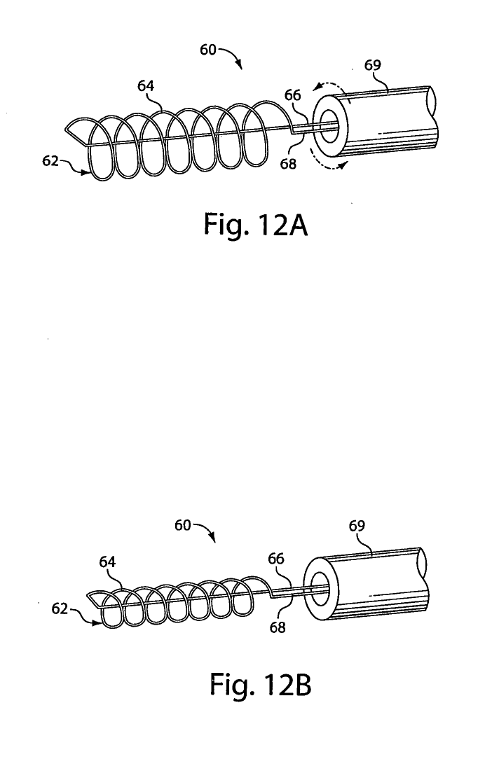

FIGS. 12A and 12B show another delivery device embodiment employing a large profile configuration to capture the stent by its inside surface and a low profile configuration that releases the stent from the delivery catheter. The spring—type delivery device 60 employs a spring coil 62 that is selectively torqued to cause the individual coils 64 to increase in diameter or decrease in diameter to engage or release the inside surface of the stent 10. The spring 62 may have torsional forces applied by opposite twisting forces applied to distal lead wire 66 and proximal lead wire 68 both extending through catheter 69 to be operated by the user. An advantage of the spring type embodiment is tat the helical configuration of the coils 64 coincide with the helical ridge of the stent to provide a secure engagement with its inside surface.

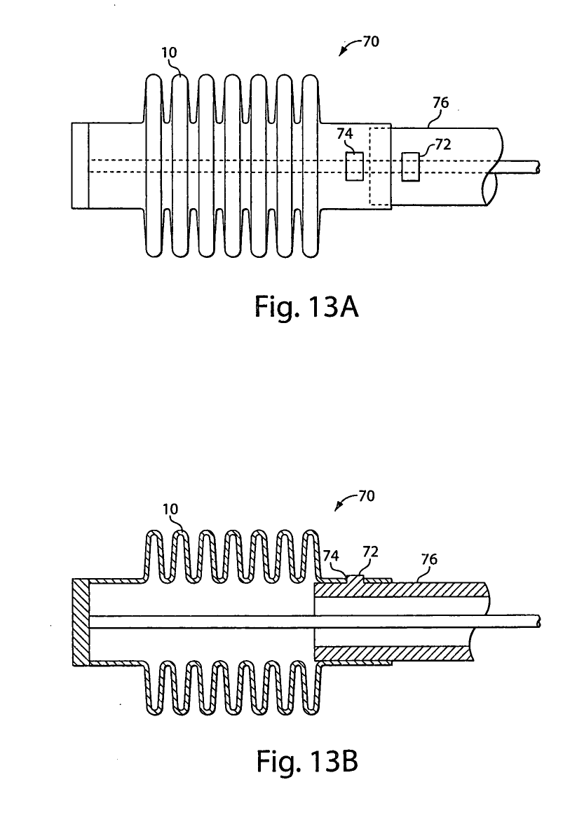

FIGS. 13A and 13B show another delivery device 70 employing a selectively engageable key 72 that can be configured to resiliently expand through catheter 76 into a keyway 74 formed in the stent 10 in order to capture the stent and move it longitudinally through the anatomy.

It should be understood that the foregoing description of the invention is intended merely to be illustrative thereof and that other modifications, embodiments and equivalents may be apparent to those who are skilled in the art without departing from its principles.

1. A delivery device for a tubular stent comprising an external surface that is flexible and selectively expandable to frictionally engage an interior surface of the tubular stent and a wedge member insertable through the external surface to cause its expansion.

2. A delivery device for a tubular stent comprising an expandable cylinder having slot along its length selectively receives a wedge shape that serves to expand the cylinder to a large profile configuration to engage an interior surface of the stent.

3. A delivery device for a tubular stent comprising an external surface that is a flexible sleeve selectively expandable to engage an interior surface of the tubular stent.

4. A delivery device for a tubular stent comprising an external surface that is a helical spring selectively expandable to engage an interior surface of the tubular stent.
